(19) United States
(12) Patent Application Publication
(10) Pub. No.: US 2008/0319394 A1
(43) Pub. Date: Dec. 25, 2008

(54) Portable infusion device with reduced level of operational noise

(75) Inventors: Ofer Yodfat, Maccabim-Reut (IL); David Leuw, Haifa (IL)

Correspondence Address: MINTZ LEVIN COHN FERRIS GLOVSKY & POPEO;ATTN: PATENT INTAKE CUSTOMER NO. 35437, ONE FINANCIAL CENTER, BOSTON, MA, 02111, US

(73) Assignee: MEDINGO, LTD., Yoqneam (IL)

(21) Appl. No.: 12/156,994

(22) Filed: Jun. 5, 2008

Related U.S. Application Data
(60) Provisional application No. 60/934,290, filed on Jun. 11, 2007.

(51) Int. Cl. A61M 5/145

(52) U.S. Cl. 604/154

(57) ABSTRACT

An infusion system, method and device for infusing therapeutic fluid into the body of a patient are provided. The device includes a driving mechanism including a plurality of gears, wherein at least one gear is adjacent to another gear. The device includes a gear in the plurality of gears having plurality of teeth and at least another gear in the plurality of gears having a plurality of teeth. The plurality of teeth of another gear interact with the plurality of teeth of the gear. At least one tooth in the plurality of teeth of the gear is elastically deformable for causing at least one tooth to elastically deform upon meshing with a tooth in the plurality of teeth of another gear and further for causing reduction of noise associated with operation of the driving mechanism.

### CROSS-REFERENCE TO RELATED APPLICATIONS

This application claims priority to U.S. Provisional Patent Application No. 60/934,290 to Yodfat et al., filed Jun. 11, 2007, and entitled “Portable Infusion Device with Reduced Level of Operational Noise” and incorporates its disclosure herein by reference in its entirety.

This application also relates to co-owned/co-pending U.S. patent application Ser. No. 11/397,115 to Yodfat et al., filed on Apr. 3, 2006, and entitled “Systems and methods for sustained medical infusion and devices related thereto”, and International Application No. PCT/IL06/001276. The disclosures of the above noted applications are incorporated herein by reference in their entireties.

### FIELD OF THE INVENTION

The present invention generally relates to systems, devices, and methods for sustained medical infusion of fluids and, in particular, to a portable infusion device. The present invention also relates to an infusion pump containing a driving mechanism that includes a motor and gears, and configured to minimize the noise produced by the driving mechanism during operation.

### BACKGROUND OF THE INVENTION

Medical treatment of some illnesses may require continuous drug infusion into various body compartments by subcutaneous and intra-venous injections, for example. Diabetes mellitus patients may require administration of varying amounts of insulin throughout the day to control their blood glucose levels. In recent years, ambulatory portable insulin infusion pumps have emerged as an alternative to multiple daily injections of insulin. These pumps, which deliver insulin at a continuous basal rate as well as in bolus volumes, were developed to liberate patients from repeated self-administered injections, and allow them to maintain a near-normal daily routine.

Conventional pumps can be either syringe-type pumps (including reservoir with a fluid propelling plunger), or peristaltic positive displacement pumps. Such conventional pumps are discussed in U.S. Pat. No. 3,631,847 to Hobbs, U.S. Pat. No. 4,498,843 to Schneider. Additionally, some pumps are also described in co-owned, co-pending U.S. patent application Ser. No. 11/397,115 to Yodfat et al., and International Patent Application No. PCT/IL06/001276, disclosures of which are incorporated herein by reference in their entireties.

Conventional driving mechanisms can be employed in the above mentioned types of pumps, and may include a DC motor, a stepper motor, a Shape Memory Alloy (“SMA”) actuator, as well as other components. The stepper motor can be accurately controlled in an open loop system and does not require a position feedback during its operation, thus, its control is less costly. Stepper motors may be activated discretely by a series of sequential input pulses (also called “pulse trains”) applied by a central processing unit (“CPU”) that controls the motion of the driving mechanism.

As stated above, the driving mechanisms of the majority of conventional infusion pumps include a motor and gears. The driving mechanisms typically include opposing gears that have teeth configured to come in contact with each other during operation of the motor. Because of that contact, the teeth of the opposing gears generate a lot of noise, which is a major drawback of the conventional driving mechanisms.

One of the causes of this noise is unavoidable tiny irregularities of the teeth that occur during a manufacturing process of the gears. In mutual meshing of gears it is desired that two opposing teeth surfaces maintain contact with each other while rotating at a predetermined angular velocity. In other words, the gears are designed so that their relative movement involves only mutual sliding along a tangential plane at the contact surfaces of the gears.

However, if the gears are eccentric, or have a pitch error or the like, their opposing teeth surfaces do not contact each other at their predetermined positions. The asymmetrical teeth interface causes uneven load distribution, subsequently causing load fluctuations and vibratory motions, and thus, generating noise.

Noise can also be attributed to a backlash. Backlash is defined as an amount by which the width of a space between two adjacent gear-teeth exceeds the width of the engaging teeth on the pitch circle. Backlash is required for reduction of friction and wear. However, it causes the opposing teeth surfaces to “hit” each other during their motion and, consequently, generate noise. Minimal or no backlash may cause gear overloading and/or overheating, and may even result in jamming and ultimately failure of the gears. This can be life threatening in drug infusion devices (e.g., insulin pumps). Therefore, some amount of backlash may be required.

When a conventional stepper motor is used, it is activated discretely by a series of pulse trains. The existence of backlash enables the teeth of the driving gear to gain speed at the beginning of each series of pulse trains and forcefully “hit” the opposing gear teeth. This further enhances the noise associated with the operation of the gears at the beginning of each series of pulse trains.

Noise is highly disturbing and unacceptable when the device is constantly attached to the body. This is especially problematic when the device is configured as an infusion pump for sustained delivery of therapeutic fluids (e.g., insulin).

The above problem has been known for a long time and various attempts to reduce noise associated with gear operation have been around for nearly a hundred years. Such attempts are discussed in U.S. Pat. No. 1,460,661, U.S. Pat. No. 3,636,792, and U.S. Pat. No. 4,127,041. These patents discuss how noise associated with gear operation can be reduced by the use of gears provided with slotted teeth. However, the above patents discuss noise reduction generally and without reference to any specific applications, or only with regard to mechanical applications, such as timepieces. The known in the art solutions for noise reduction do not address such an application as pumps for the delivery of therapeutic fluids.

### SUMMARY OF THE INVENTION

The present invention relates to a system and a method for reducing noise in a medical device, such as an insulin pump (or a miniature insulin pump), during operation of the medical device and while the device is configured to be connected to the body of a patient. In some embodiments, the present invention is a miniature portable programmable skin adherable infusion device that operates quietly. The present invention allows minimizing the noise associated with operation of gear(s) in the infusion device. In some embodiments, the infusion device includes a two-part dispensing unit provided with gears designed for minimizing the noise associated with gear operation, while maintaining gear durability, reliability and efficiency. In some embodiments, the present invention provides a simple and low-cost solution for minimizing the noise associated with gear operation.

In some embodiments, the present invention's device for medical infusion containing a dispensing unit is thin and can be attached to the body of a patient at any desired location. The two-part dispensing unit has a reusable part and a disposable part. The reusable part includes electronics and at least a portion of a driving mechanism, and the disposable part includes a reservoir that is configured to contain a therapeutic fluid.

In some embodiments, the present invention is a dispensing unit with a gear train based driving mechanism configured to reduce noise during its operation. The dispensing unit can be a miniature dispensing unit. The driving mechanism can be configured to reduce noise while maintaining durability, efficiency, and functional reliability of the miniature-size gears. The solution for noise reduction according to the present invention is inexpensive.

Some embodiments of the present invention relate to a device that can deliver therapeutic fluid to the body of a patient. The fluid delivery device according to the present invention can include three units: a dispensing unit, a skin-adherable needle unit and a remote control unit. The dispensing unit may employ different dispensing mechanisms, such as a syringe-type reservoir with a propelling plunger or a peristaltic positive displacement mechanism. The dispensing unit can be connected to and disconnected from the skin adherable needle unit upon patient discretion. A remote control unit communicates with the dispensing unit and allows programming of the therapeutic fluid delivery, user input and data acquisition.

In some embodiments, the dispensing unit can include two parts: a reusable part and a disposable part. The disposable part includes a reservoir and an outlet port. The reusable part includes electronics and at least a portion of the driving mechanism.

In some embodiments, the driving mechanism can be a part of a pumping mechanism. The driving mechanism includes a stepper motor and gear trains. The driving mechanism includes opposing gears having teeth. The teeth of the opposing gears come in contact with each other during operation of the driving mechanism and, thus, generate noise. In order to minimize the noise generated by the teeth of the opposing gears, some embodiments of the present invention include gears that can be provided with slots. The slots are formed between two adjacent gear teeth or in the gear teeth. Alternatively, the gears can be provided with apertures that are formed in each one of the gear teeth. The slots/apertures allow the gear teeth to undergo some elastic deformation when contacting the teeth of opposing gears and, hence, minimize the produced noise.

### BRIEF DESCRIPTION OF THE DRAWINGS

FIGS. 1a-c illustrate an exemplary single-part dispensing unit, an exemplary two-part dispensing unit and an exemplary remote control unit, according to some embodiments of the present invention.

FIGS. 2a-b illustrate an exemplary single-part dispensing unit, an exemplary two-part dispensing unit, an exemplary remote control unit and an exemplary needle unit, according to some embodiments of the present invention.

FIGS. 3a-b illustrate an exemplary needle unit having a cradle base, a well and a cannula, according to some embodiments of the present invention.

FIG. 4 illustrates the dispensing unit and the needle unit prior to being connected, according to some embodiments of the present invention.

FIGS. 5a-c illustrate an exemplary connection between the dispensing unit and the needle unit, according to some embodiments of the present invention.

FIGS. 6a-c illustrate an exemplary dispensing unit being directly adhered to the skin of a patient, according to some embodiments of the present invention.

FIGS. 7a-b illustrate an exemplary single-part dispensing unit (illustrated in FIG. 7a) and an exemplary two-part dispensing unit (illustrated in FIG. 7b) that employ a peristaltic pumping mechanism, according to some embodiments of the present invention.

FIG. 8 illustrates exemplary components of a reusable part of a dispensing unit that employs a peristaltic pumping mechanism, according to some embodiments of the present invention.

FIG. 9 illustrates an exemplary driving mechanism of the dispensing unit illustrated in FIG. 8.

FIG. 10 illustrates exemplary components of a syringe-type dispensing unit, according to some embodiments of the present invention.

FIGS. 11a-11c and 12a-12b illustrate exemplary gears provided with slots located between gear teeth, according to some embodiments of the present invention.

FIGS. 13a-c illustrate an exemplary gear provided with slots that are cut through the gear teeth, according to some embodiments of the present invention.

FIGS. 14a-b illustrate an exemplary gear provided with apertures that are formed in the gear teeth, according to some embodiments of the present invention.

### DETAILED DESCRIPTION OF THE INVENTION

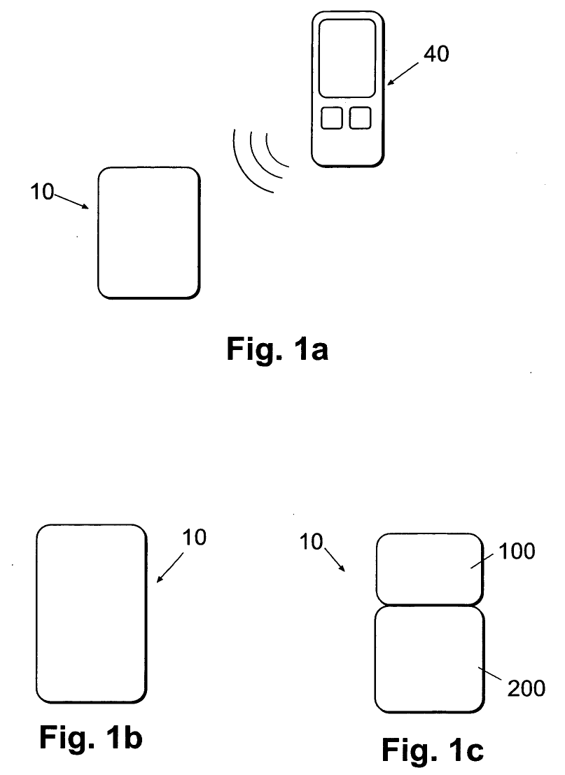

FIG. 1a illustrates an exemplary fluid delivery device having a dispensing unit (10) and a remote control unit (40), according to some embodiments of the present invention. In some embodiments, the dispensing unit (10) can be configured to include a single part (shown in FIG. 1b) or two parts (shown in FIG. 1c). In some embodiments, the dispensing unit (10) can be configured to include a reusable part (100) and a disposable part (200). In the two-part embodiment, the dispensing unit includes a reusable part (100) and a detachably-connectable disposable part (200). The remote control unit (40) communicates with the dispensing unit (10) and includes a display, control button(s), a processor, a memory, and any other components for communicating with the unit (10). The remote control unit (40) can communicate with the unit (10) using wired, wireless, RF, or any other suitable methods of communication. The remote control unit (40) can be any conventional remote control means, e.g. a cellular telephone, an iPod, a PDA, or any other suitable device.

In some embodiments, the remote control unit (40) can include various controls, a processor, and communications capabilities that can interact and control operation of the dispensing unit (10). The remote control unit (40) can also include a display screen that can display status and other information for the patient (the terms “patient” and “user” are used in this description). The dispensing unit (10) can also include various controls, a processor, and communication capabilities in addition to other components, which are described below. The dispensing unit's (10) components interact with the remote control unit (40) components for operational purposes.

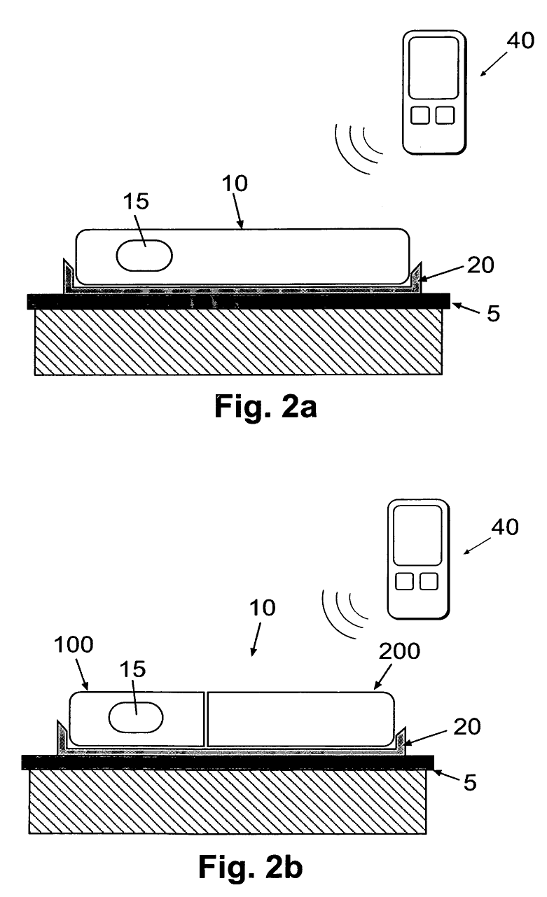

FIG. 2a illustrates an exemplary fluid delivery device having a single-part dispensing unit (10), a needle unit (20) and the remote control unit (40), according to some embodiments of the present invention. The dispensing unit (10) can be configured to be connected to the needle unit (20) subsequent to the needle unit (20) being adhered to the skin (5) of a patient. In some embodiments, the adherable to the skin part of the needle unit (20) can also be referred to as a cradle unit. The dispensing unit (10) may be reconnected to or disconnected from the needle unit (20) upon discretion of the patient. Fluid delivery can be programmed by the remote control unit (40) or manually by at least one button (15) provided on the dispensing unit (10). In some embodiments, the needle unit (20) can include an adhesive layer that allows the needle unit (20) to be adhered to the skin (5) of the patient.

FIG. 2b illustrates an exemplary fluid delivery device having a two-part dispensing unit (10), the needle unit (20) and the remote control unit (40), according to some embodiments of the present invention. As stated above, the two-part dispensing unit (10) includes the reusable part (100) and the disposable part (200). The parts (100) and (200) are connected to each other prior to coupling with the needle unit (20).

In an exemplary embodiment the reusable part (100) includes manual buttons (15) for controlling the operation of the dispensing unit (10). Referring to FIGS. 2a-b, the needle unit (20) can include at least one latch (or any other securing mechanism) for securely coupling the dispensing unit (10) to the needle unit (20). As can be understood by one skilled in the art, fluid delivery can be implemented using any fluid delivery device, including those shown in FIGS. 2a-2b.

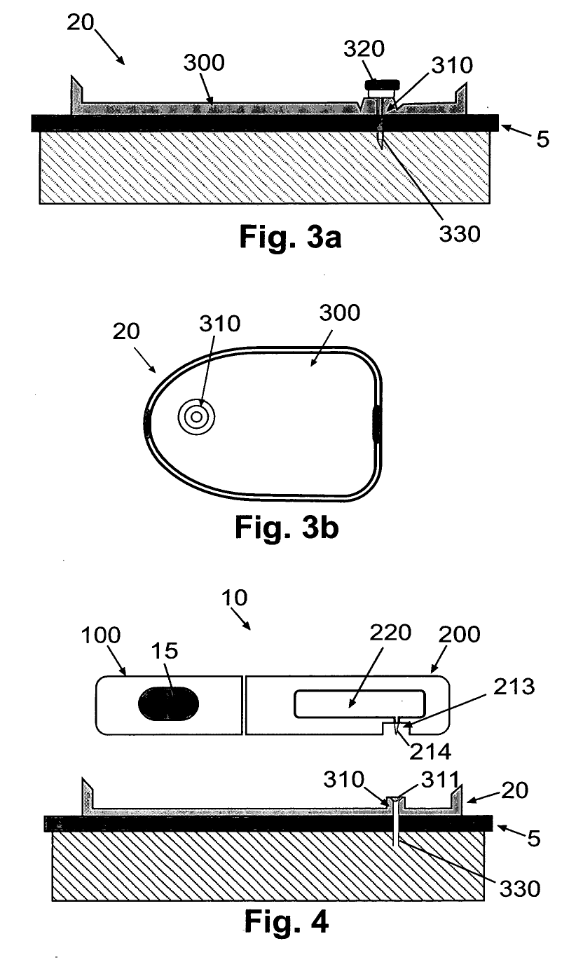

FIGS. 3a-b are side and upper views, respectively, of the needle unit (20) associated with the dispensing unit (not shown in FIGS. 3a-b). In some embodiments, the needle unit (20) can be configured to include a cradle base (300), a cannula (330), a penetrating member (320), and a well (310).

The cradle base (300) is initially adhered to the skin (5) of the patient. The cradle base (300) includes an adhesive layer that allows the patient to adhere the cradle base (300) to the skin (5). On the cradle base (300) is located a well portion (310) that allows insertion of the penetrating member (320) along with the cannula (330). The penetrating member (320) is configured with a sharp end that pierces the skin (5) of the patient, thereby allowing subcutaneous insertion of the cannula (330). The penetrating member (320) can be removed from the skin (5) after insertion of the cannula (330). The well portion (310) provides a fluid-tight conduit for delivery of therapeutic fluid from the dispensing unit to the patient via the cannula (330).

In some embodiments, the cradle base (300) can be a sheet having an adhesive layer that faces the skin (5) of the patient. In some embodiments, the cradle base (300) can be configured to include a securing means (e.g., latch(es), snap-fit device(s)) for detachably securing the dispensing unit to the needle unit.

In some embodiments, the well (310) can be a tubular protrusion extending upwardly from the cradle base (300). The well (310) can be further configured to allow anchoring of the cannula (330) thereto (using, for example, latches, snap-fit devices, etc) and alignment of the outlet port of the dispensing unit with the cannula to provide proper fluid delivery from the dispensing unit to the body of the patient.

As can be understood by one skilled in the art, there are various options for attaching the needle unit (20) and its components to the body of the patient. Some of these options are described in the co-owned, co-pending U.S. Provisional Patent Application 60/876,679, filed Dec. 22, 2006, U.S. patent application Ser. No. 12/004,837, filed Dec. 20, 2007, and International Patent Application No. PCT/IL07/001,578, filed Dec. 20, 2007, the disclosures of which are incorporated herein by reference in their entireties.

FIG. 4 illustrates the two-part dispensing unit (10) being coupled to the needle unit (20), according to some embodiments of the present invention. The dispensing unit (10) includes a reservoir (220) for therapeutic fluid, an outlet port (213) and a connecting lumen (214). The connecting lumen (214) maintains fluid communication between the reservoir (220) and the outlet port (213) of the dispensing unit. Upon connecting the dispensing unit (10) and the needle unit (20), the connecting lumen (214) pierces a septum (311) configured to provide sealing of the well (310). Upon removal of the connecting lumen (214), the septum (311) re-seals the well (310). Referring back to FIG. 4, upon insertion of the connecting lumen (214), the lumen (214) fluidly connects the outlet port (213) of the dispensing unit (10) and the cannula (330), thereby allowing fluid delivery via the cannula (330) to the subcutaneous compartment. The outlet port (213) allows repetitive connection and disconnection of the dispensing unit (10) to and from the needle unit (20).

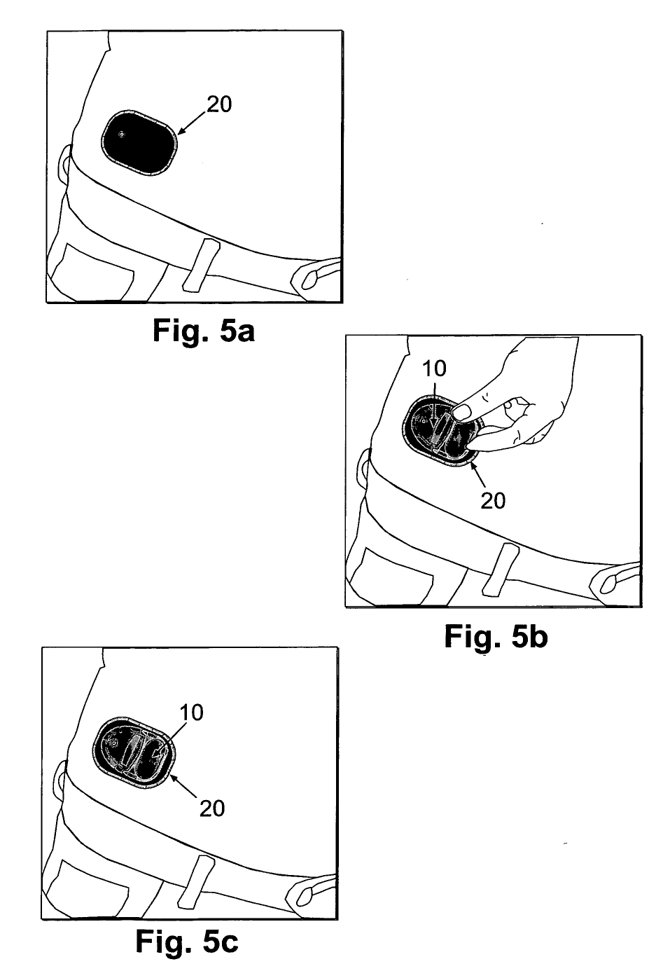

In some embodiments (including those illustrated in FIGS. 5a-c), the needle unit (20) can be first adhered to the skin of the patient and then the dispensing unit (10) can be connected to and disconnected from the needle unit (20) upon discretion of the patient. FIG. 5a illustrates the needle unit (20) adhered to the skin of the patient. FIG. 5b illustrates a connection between the dispensing unit (10) and the needle unit (20) adhered to the skin of the patient. FIG. 5c illustrates the dispensing unit (10) after it has been connected to the needle unit (20) and being ready for operation.

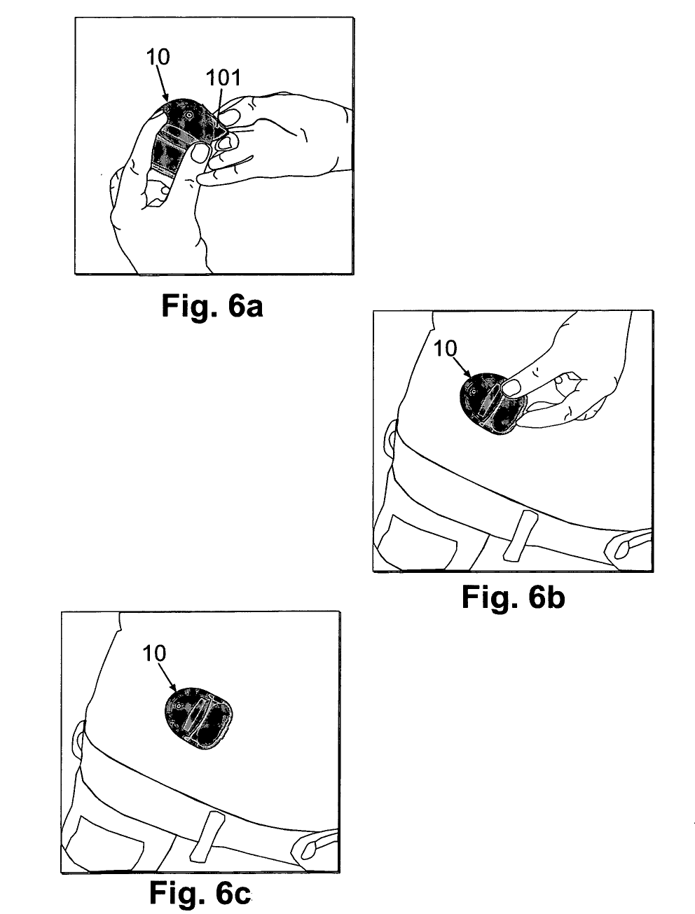

FIGS. 6a-c illustrate other exemplary embodiments of the present invention where the dispensing unit (10) is configured to be directly adhered to the skin (5) of the patient. FIG. 6a illustrates peeling of the adhesive protective sheet (101) from the lower face of the dispensing unit (10). FIG. 6b illustrates the dispensing unit (10) adhered to the skin of the patient. FIG. 6c illustrates the dispensing unit (10) adhered to the skin of the patient and being ready for operation.

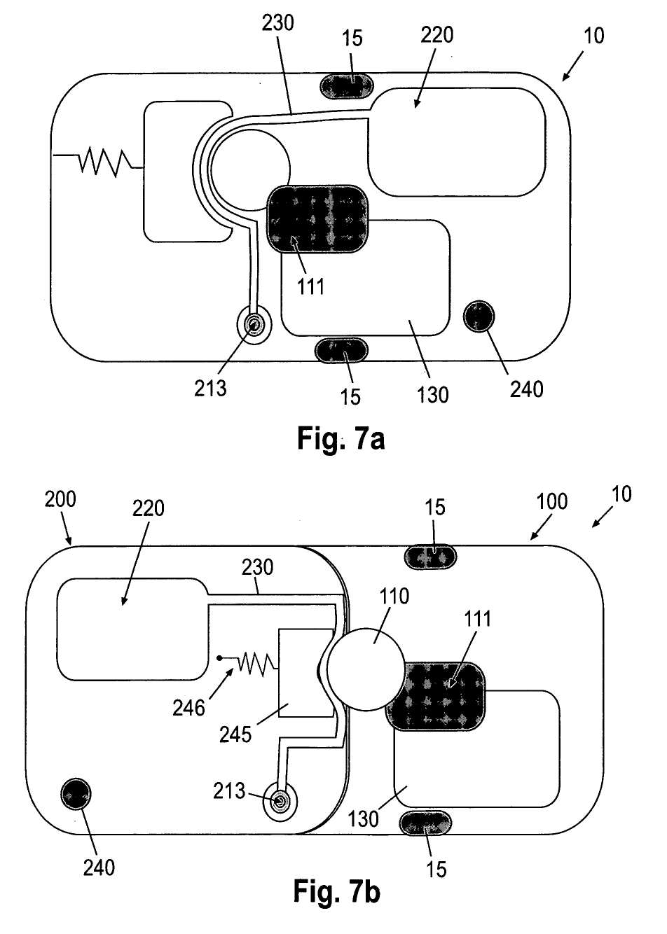

FIG. 7a illustrates the dispensing unit (10) disposed within a single housing and having a peristaltic pumping mechanism. The dispensing unit (10) includes a reservoir (220) for therapeutic fluid, a fluid delivery tube (230), the outlet port (213), electronic components (130), a battery (240), a driving mechanism (111), and buttons (15). The reservoir (220) is in fluid communication with the outlet port (213) via the fluid delivery tube (230). The electronic components (130) are coupled to the driving mechanism (111) that further actuates the pump, thereby causing dispensing of the therapeutic fluid from the reservoir (220) through the fluid delivery tube (230) to the outlet port (213). In some embodiments, a driving mechanism (111) includes a stepper motor, a DC motor, a SMA actuator, or the like. The electronic components (130) can be disposed on a printed circuit board (“PCB”) An energy supply means (240) provide power to the electronic components (130) and the driving mechanism (111), where the energy supply means can be one or more batteries. Control buttons (15) and/or remote control unit (40) can perform programming of fluid dispensing. Such programming can involve providing instructions as to whether and when to dispense an appropriate dosage (e.g., bolus, basal, etc.) of therapeutic fluid. In some embodiments, therapeutic fluid is insulin.

FIG. 7b illustrates the two-part dispensing unit (10) having the reusable part (100) and the disposable part (200), wherein each part is contained in a separate housing, according to some embodiments of the present invention. The reusable part (100) includes a rotary pump wheel (110), the driving mechanism (111), the electronic components (130) and at least one manual button (15). The rotary pump wheel (110) is connected to the driving mechanism (111), which upon receipt of appropriate signals from the electronic components (130) causes rotation of the rotary pump wheel (110), thereby causing displacement of therapeutic fluid within the fluid delivery tube (230).

The disposable part (200) includes the reservoir (220), the delivery tube (230), the energy supply means (240), and the outlet port (213). Additionally, the disposable part (200) includes a stator (245) elastically supported by a spring (246). Fluid dispensing is possible after connecting the reusable part (100) to the disposable part (200). Upon connection of the two parts, the energy supply means (240) becomes coupled to the electronic components (130), thereby providing power to the components (130). As such, the unit (10) becomes operational causing the driving mechanism (111) to cause rotation of the rotary wheel (110). Further, the fluid delivery tube (230) becomes disposed between rollers of the rotary wheel (110) and the stator upon connection of the two parts. As the rotary wheel (110) rotates, its rollers compress the tube (230) against the stator, thereby displacing the fluid within the delivery tube (230) from the reservoir (220) towards the outlet port (213). An exemplary discussion of fluid dispensing mechanisms, systems and methods is found in a co-owned, co-pending U.S. patent application Ser. No. 11/397,115 and International Patent Application No. PCT/IL06/001276, the disclosures of which are incorporated herein by reference in their entireties.

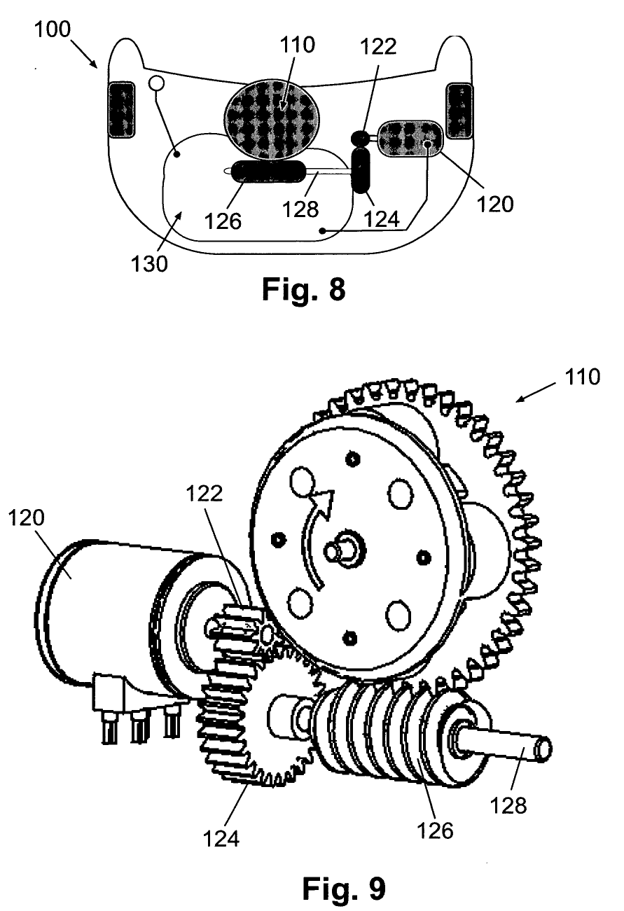

FIG. 8 illustrates an exemplary reusable part (100), according to some embodiments of the present invention. The reusable part (100) includes, among other components discussed above, the rotary pump wheel (110), a motor (120), a first gear (122), a secondary gear (124) and a worm (126). The motor (120) is electrically coupled to the electronic components (130) which provide instructions to the motor (120). The motor (120) is further coupled to the first gear (122). The gear (122) includes a plurality of teeth, which interact with the plurality of teeth of the secondary gear (124). The gear (124) is disposed on a shaft (128). The worm (126) is also disposed on the shaft (128). The worm (126) interacts with teeth of the rotary wheel (110) during rotation. As can be understood by one having ordinary skill in the art, the secondary gear (124) and the worm (126) can be manufactured as a single unit or part to be carried by the shaft (128).

FIG. 9 illustrates an exemplary driving mechanism (111) and rotary pump wheel (110) of the dispensing unit configured as a peristaltic pump, according to some embodiments of the present invention. Upon receipt of appropriate signals from the electronic components (130), the motor (120) rotates the first gear (122), the plurality of teeth of which is meshed with the plurality of teeth of the secondary gear (124). Thus, rotation of the first gear (122) in one direction causes rotation of the secondary gear (124) in an opposite direction. The rotational momentum is thus transferred to the secondary gear (124). Since the secondary gear (124) is disposed on the same shaft as the worm (126), rotation of the secondary gear (124) causes rotation of the worm (126). The teeth of the worm (126) are further meshed with the teeth of the rotary wheel (110).

An example of the driving mechanism (111) components is disclosed in the co-owned/co-pending U.S. Patent Provisional Application No. 60/928,751, filed May 11, 2007, International Patent Application No. PCT/IL08/000,642, filed May 11, 2008, both entitled “Methods and Apparatus for Monitoring Rotation of an Infusion Pump Driving Mechanism” and U.S. Patent Provisional Application No. 60/928,815, filed May 11, 2007, International Patent Application No. PCT/IL08/000,641, filed May 11, 2008, both entitled “A Positive Displacement Pump”, the disclosures of which are incorporated herein by reference in their entireties.

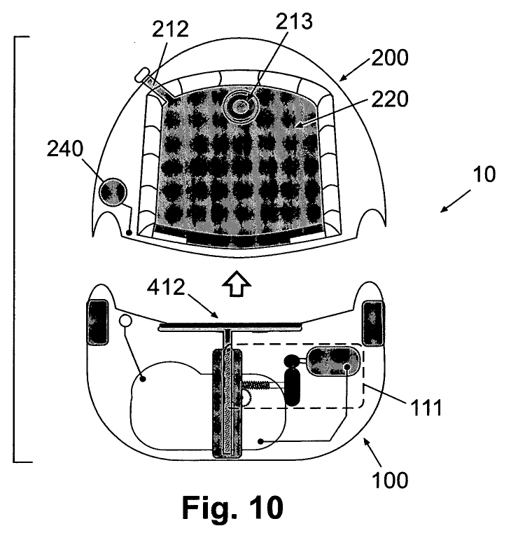

FIG. 10 illustrates another exemplary embodiment of the two part-dispensing units (10), where the dispensing unit is configured as a syringe-type pump. In this embodiment, the reusable part (100) includes the driving mechanism (111) provided with a piston (412), and the disposable part (200) includes a syringe-type fluid reservoir (220), an inlet port (212) and the outlet port (213). This exemplary configuration is disclosed in the co-owned/co-pending U.S. Provisional Patent Application No. 60/928,750 to Yodfat et al., filed on May 11, 2007, and International Patent Application No. PCT/IL08/000,643, filed May 11, 2008, both entitled “Fluid Delivery Device”, the disclosures of which are incorporated herein by reference in their entirety. As previously stated, fluid dispensing is possible after connecting the reusable part (100) to the disposable (200) part. Rotational momentum is produced by the driving mechanism (111), as discussed above with reference to FIG. 9. In the embodiment shown in FIG. 10, the rotational momentum is transferred into a linear movement of the piston (412) of the syringe-type pump. In this embodiment, the dispensing unit (10) employs a driving mechanism having gears having a plurality of teeth, where teeth of adjacent gears are meshed together. During operation of the driving mechanism, the teeth of adjacent gears come in contact with each other.

Upon contact of the teeth of adjacent gears, whether in the embodiment shown in FIG. 9 or FIG. 10, teeth contact may generate noise. Noise may be generated when opposite teeth surfaces “hit” each other during rotating motion.

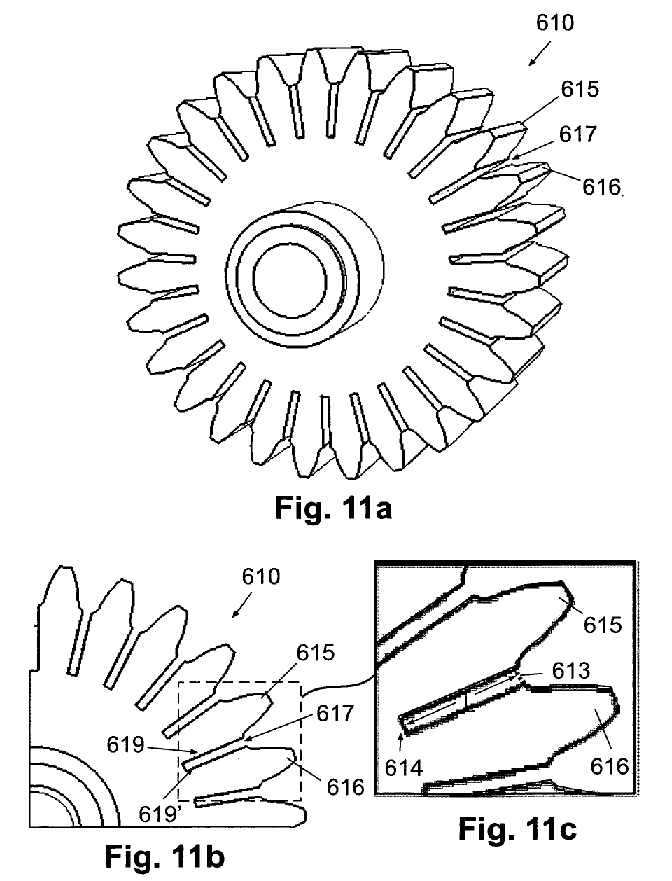

FIG. 11a illustrates an exemplary gear (610) in a driving mechanism of the dispensing unit (10), according to some embodiments of the present invention. The gear (610) includes a plurality of teeth separated by a plurality of elongated radial slots (617), disposed between two adjacent gear teeth. As illustrated in FIG. 11a, a slot (617) is disposed between adjacent gear teeth (615), (616). The slots are thus disposed in a circumferential fashion around the gear (610). Providing of the slot imparts the gear teeth with an added degree of elasticity. As such, the gear teeth are slightly elastically deformable upon contact with each other. This means that as one gear tooth of one gear comes in contact with another gear tooth of another gear, the gear teeth flex and/or bend hence, alleviating the clunking noise associated with gear teeth contact. The degree of flexibility/bendability of each gear tooth can be varied based on desired specification of the system.

In some embodiments, the gear (610) can be fabricated from a suitable plastic material, such as Polyoxymethylene (e.g., DELRIN®, manufactured by DuPont, USA), the mechanical properties of which allow the desired elastic deformation. In some embodiments, the gear (610) can be designed to be relatively thick, so that the elastic deformation does not compromise the strength and durability of the gear teeth. Such design of gear teeth in miniature-sized fluid dispensing devices is not trivial and provides an unconventional approach to solving noise related problems in therapeutic fluid dispensing devices, as many conventional devices suffer from excessive noise generated during their operation.

FIG. 11b illustrates an enlarged portion of the gear (610) shown in FIG. 11a. As illustrated, the radial slots are configured such that the two lateral faces (619), (619′) of each slot (617) are parallel to each other. As can be understood by one skilled in the art, the lateral faces of each slot (617), instead of being parallel to each other (i.e., forming a rectangular slot (617)), can be not parallel to each other. For example, the faces (619), (619′) can form a slot (617) having a V-shape or any other desired shape (e.g., the faces (619), (619′) can be disposed at any angle with regard to each other).

Based on the desired configuration, the slot (617) can have a predetermined width and length. The length and width of the slots, can be determined according to: (1) the maximum force, which could be potentially applied to the gear, (2) the yield point of the material from which the gear is fabricated (i.e., the stress at which the material begins to plastically deform), (3) the desired degree of elasticity, or any other factors. The above noise reduction approach, as well as other described noise reduction solutions, can be applied to gears of all sizes, large and small alike. In some embodiments, for example in a miniature-size gear, the approximate slot width is equal to 0.1 millimeters (“mm”), and the approximate slot length is equal to 0.7 mm. In some embodiments, the gear can be configured to have 27 teeth, an outer diameter of 5.8 mm, and module (“m”)=0.2 mm. The term module refers to a parameter used in the field of spur wheels and represents a ratio of a pitch diameter (“D”) to the number of teeth in the gear (“N”) (for example, the module of a gear having 27 teeth and a pitch diameter of 5.4 mm is:

$$m = \frac{D}{N} = \frac{5.4}{27} = 0.2 mm$$

). The pitch diameter D is a commonly used term in mechanical engineering and refers roughly to the diameter of the circle which passes through the center of the gear teeth. The nominal gear size is usually the pitch diameter. As can be understood by one skilled in the art, the gear can include any number of teeth, any outer diameter, as well as its module parameters. In some embodiments, once the number of teeth and the module parameter in the gear are defined, other characteristics of the gear can be defined (some embodiments can exclude determination of width/thickness). It will be noted that, in this embodiment, the slot length L is defined as the distance between the center of the root of the tooth (613) and the base of the slot (614), as demonstrated in FIG. 11c. As can be understood by one having ordinary skill in the art, the dimensions of each slot (617) can vary according to the desired configuration of the system.

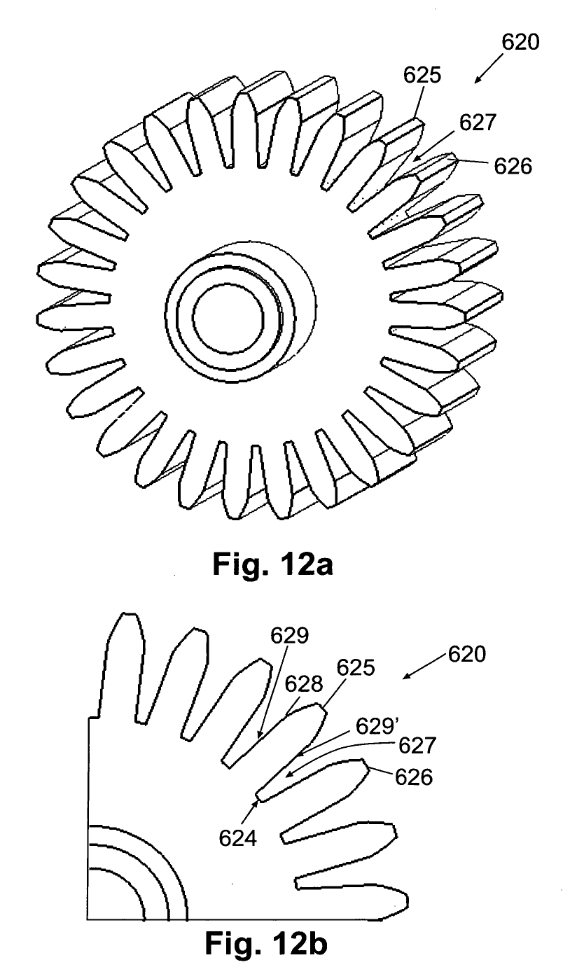

FIGS. 12a-b illustrate another exemplary gear (620) provided with slots (627) between adjacent gear teeth, according to some embodiments of the present invention. In this embodiment, the slots are configured such that their lateral faces (629), (629′) are not parallel to each other and they are further configured to increase the space between two adjacent gear teeth (625), (626), thus, providing additional flexibility to the gear teeth.

FIG. 12b illustrates an enlarged fragment of the gear (620). In some embodiments of miniature-sized gears (e.g., 27-tooth gear, module 0.2 mm, outer diameter 5.8 mm), the approximate width of the slot (627) at its base (624) equals to 0.1 mm and the approximate length of the slot (627) equals to 0.7 mm. In some embodiments, within the same gear, the lateral faces (629) of adjacent teeth can be parallel to each other or disposed non-parallel to each other. Further, the lateral faces (629) of one tooth can be parallel to each other and another face (629) of another gear tooth can be disposed non-parallel to the first face (629) of the first gear tooth. Additionally, the parallel/angular disposition of facet can alternate with respect to each other. As can be understood by one skilled in the art, other arrangements of teeth are possible. In gears provided with an involute profile, the slots can be configured to originate at the end of the involute (628), as illustrated in FIGS. 12a-b, to ensure that the efficiency and functional reliability of the gear (620) are not compromised.

The slots and the elasticity of the teeth further allow greater tolerance to the defects and/or inaccuracies in the manufacturing process of the gears and in the assembly of the driving mechanism. This allows better coupling and assembly of the gears in the system, according to some embodiments of the present invention.

For example, a driving mechanism can include a first gear (outer diameter of about 2.2 mm and pitch diameter of about 1.8 mm) coupled to a secondary gear (outer diameter of about 5.8 mm and pitch diameter of about 5.4 mm) and the gears are positioned such that their centers are located 3.63 mm apart, i.e., the distance between their shafts is 3.63 mm (this distance between the shafts includes a tolerance of 0.03 mm) In some embodiments of the present invention, providing slots between adjacent gear teeth as illustrated in FIGS. 12a-12b, allows a larger tolerance which can be on the order of approximately 0.13 mm, without increasing the distance between the shafts which would require an enlargement of the dispensing unit. As such, the systems of the present invention are less sensitive to the manufacturing defects in the gears and in the assembly of the driving mechanism. As can be understood by one skilled in the art, other distances/tolerances' parameters are possible.

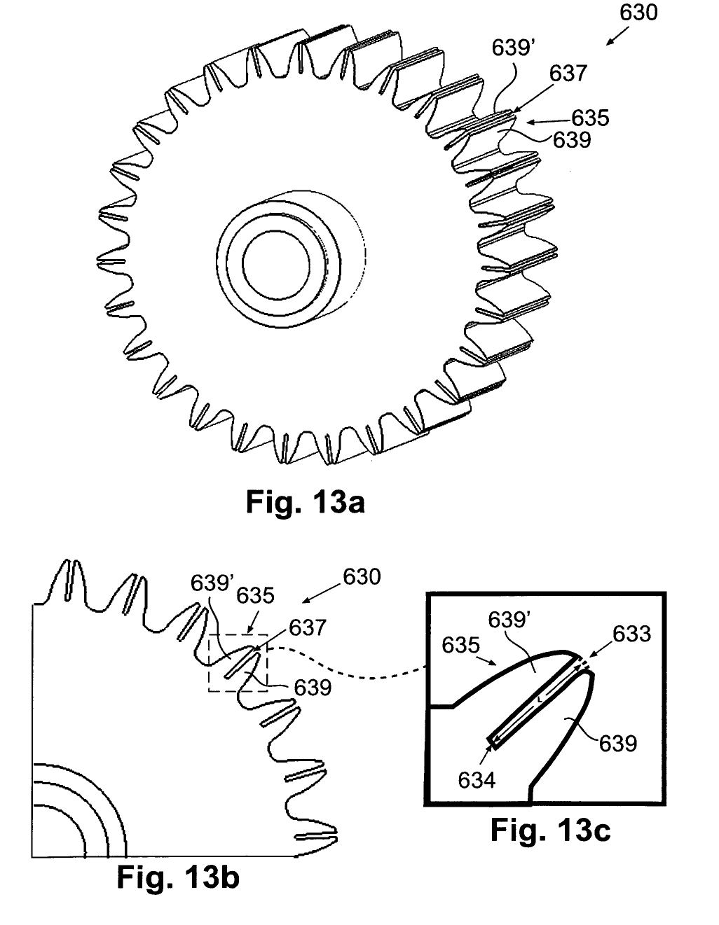

FIG. 13a illustrates another exemplary gear (630) provided with slots (637) cut through the center of each gear tooth (635) in the radial direction, according to some embodiments of the present invention. In this embodiment, the slots are disposed in the gear teeth themselves rather than between the gear teeth, as shown in FIGS. 12a-b. The slot (637) divides the gear tooth (635) into two portions (639) and (639′), which are capable of bending and flexing in different directions as the gear (630) rotates and its teeth interact with teeth of other gears. The portions (639) and (639′) are able to compress toward each other, bend in the same direction, or bend in opposite directions. One of the advantages of the slotted gear teeth is that they are elastically deformable and able to absorb shock and vibrations by undergoing an elastic deformation, thus, minimizing the noise associated with gear operation.

FIG. 13b illustrates an enlarged fragment of the gear (630). In some embodiments of the miniature-size gears (e.g., 27-tooth gear, module 0.2 mm, outer diameter 5.8 mm), the width of the slot (637) is approximately equal to 0.05 mm. The length of the slot (637) can vary according to the desired degree of elasticity. As can be understood by one having skill in the art, the length L of the slot (637) can be defined as the distance between the center of the top of the tooth (633) and the base of the slot (634). The slot (637) can reach the root of the gear tooth, or it can otherwise be longer or shorter than the gear tooth. As can be understood by one skilled in the art, various other slots can be used to achieve elastic deformation, e.g., a pair of slots cut through the tooth and shaped as an inverted “V”. The configuration of slots (637) throughout the gear (630) can vary from tooth to tooth, e.g., some slots can be rectangular, some can be V-shaped, and some can have any other desired shape. Further, width and length of each slot (637) in the gear can be the same or can vary from tooth to tooth.

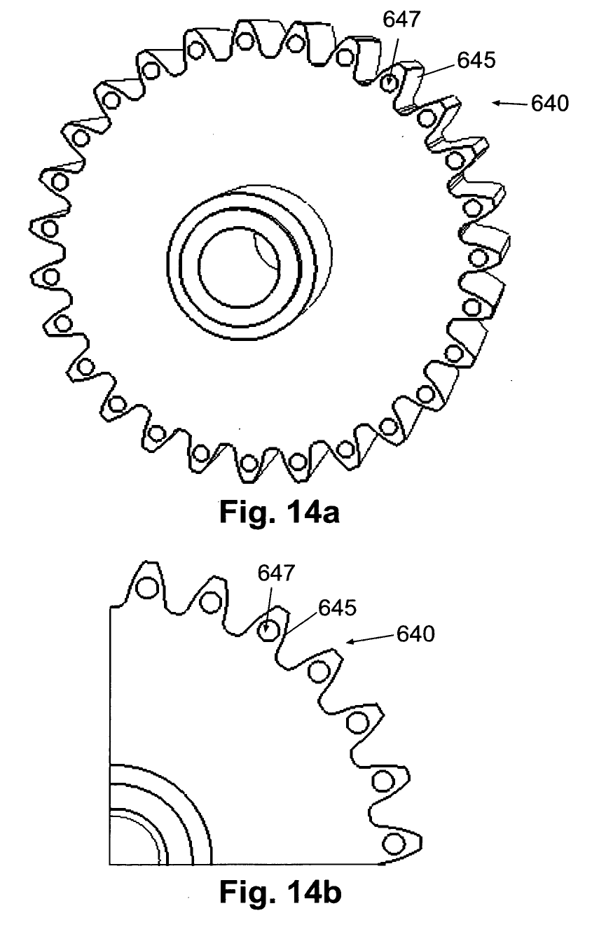

FIG. 14a illustrates yet another exemplary gear (640) provided with apertures (647) in each gear tooth (645), according to some embodiments of the present invention. The apertures (647) are configured to enable the gear teeth to absorb shock and vibration by undergoing elastic deformation, thus, minimizing the noise associated with gear operation.

FIG. 14b is an enlarged view of the gear (640). In some embodiments of the miniature-size gears (e.g., 27-tooth gear, module 0.2 mm, outer diameter 5.8 mm), an exemplary diameter of such aperture (647) can be in the range from about 0.1 mm to about 0.2 mm. The apertures may be round, as illustrated, oval, square, rectangular, polygonal, multi-sided or any other desired shape. However, the shape of the apertures, as well as the dimensions of the apertures can be determined according to the abovementioned considerations. As can be understood by one skilled in the art, the sizes, shapes and disposition of the apertures (647) can vary from tooth to tooth within the gear (640).

In some embodiments, another solution for minimizing the noise associated with gear operation employs helical gears (i.e., gears the teeth of which are cut at an angle to the face of the gear) instead of spur gears. The helix angle of one gear should be negative with respect to the helix angle of the adjacent gear. The engagement of the teeth of helical gears is more gradual than that of the teeth of spur gears, thus helical gears run more smoothly and quietly than spur gears.

As can be understood by one having ordinary skill in the art, the present invention's system for reducing operating noise in fluid dispensing systems can be employed to reduce noise in spur gears, helical gears, double helical gears, bevel gears, crown gears, hypoid gears, worm gears, rack and pinion gears, sun and planet gears, and other types of gears.

Other embodiments may include different combinations of the noise reduction solutions described hereinabove, e.g., a combination between the embodiments described in connection with FIGS. 11a-c and 13a-c (slots between the gear teeth combined with slots in each gear tooth), or a combination between the embodiments described in connection with FIGS. 12a-b and 14a-b (slots between the gear teeth combined with apertures in each gear tooth), etc. As can be understood by one skilled in the art, combinations of all slots, apertures, and variable-angle faces of the slots in gear teeth can be also employed to further reduce the noise.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, presently unclaimed inventions are also contemplated. The inventors reserve the right to pursue such inventions in later claims.

What is claimed:

1. An infusion device for infusing therapeutic fluid into a body of a patient, comprising:
a driving mechanism provided with a plurality of gears, wherein at least one gear is adjacent to another gear, said driving mechanism having
a gear in the plurality of gears having plurality of teeth;
another gear in the plurality of gears having a plurality of teeth, said plurality of teeth of said another gear interact with said plurality of teeth of said gear;
at least one tooth in said plurality of teeth is configured to be elastically deformable upon interacting with a tooth in said plurality of teeth of said another gear so as to cause reduction of noise associated with operation of the driving mechanism.

2. The infusion device according to claim 1, wherein at least one slot is provided between adjacent gear teeth in said plurality of teeth to provide elasticity to said at least one tooth.

3. The infusion device according to claim 2, wherein said slot is defined by two faces disposed oppositely to each other.

4. The infusion device according to claim 3, wherein said faces are parallel to each other.

5. The infusion device according to claim 3, wherein said faces are non-parallel to each other.

6. The infusion device according to claim 1, wherein at least one slot is provided within at least one tooth of said plurality of teeth.

7. The infusion device according to claim 6, wherein said at least one slot divides said tooth into two tooth parts.

8. The infusion device according to claim 7, wherein said at least one slot is defined by two faces disposed oppositely to each other.

9. The infusion device according to claim 8, wherein said faces are parallel to each other.

10. The infusion device according to claim 8, wherein said faces are non-parallel to each other.

11. The infusion device according to claim 1, wherein at least one aperture is provided in at least one tooth of said plurality of teeth to provide elasticity to said at least one tooth.

12. The infusion device according to claim 11, wherein said aperture has a shape selected from a group consisting of a round, oval, square, rectangular, polygonal, and multi-sided shape.

13. The infusion device according to claim 2, further comprising at least one slot provided in the at least one tooth.

14. The infusion device according to claim 2, further comprising at least one aperture provided in the at least one tooth.

15. The infusion device according to claim 1, wherein said infusion device comprises a dispensing unit provided with a housing for accommodating at least a portion of the driving mechanism.

16. The infusion device according to claim 15, wherein said dispensing unit comprises:
a reusable part having at least a portion of said driving mechanism and electronic components;
a disposable part having a reservoir; and,
an energy supply means electrically coupled to at least one of said electronic components for supplying energy upon connection of said reusable part and said disposable part.

17. The infusion device according to claim 15, wherein said dispensing unit is configured as a peristaltic pump.

18. The infusion device according to claim 15, wherein said dispensing unit is configured as a syringe pump.

19. The infusion device according to claim 17, wherein said dispensing unit is provided with a rotary pump wheel;
said driving mechanism further includes a motor, a worm having a plurality of worm teeth, and said plurality of gears includes a first gear having plurality of teeth and a secondary gear having plurality of teeth;
said motor is configured to rotate said first gear;
said plurality of teeth of the first gear are configured to be meshed with said plurality of teeth of the secondary gear;
said plurality of teeth of the secondary gear are configured to be meshed with said plurality of the worm teeth; and
said worm is configured to rotate said rotary pump wheel.

20. The infusion device according to claim 15, in which said dispensing unit is attachable to and detachable from the body of the patient via a needle unit.

21. The infusion device according to claim 15, further comprising a remote control unit for communicating with the dispensing unit.

22. The infusion device according to claim 1, wherein said gears are configured to be used in miniature-size pumps.

23. The infusion device according to claim 1, wherein said gear and said another gear are selected from a group consisting of spur gears, helical gears, double helical gears, bevel gears, crown gears, hypoid gears, worm gears, rack and pinion gears, and sun and planet gears.

24. An infusion device for infusing therapeutic fluid into a body of a patient, comprising:
a driving mechanism provided with a plurality of gears, wherein at least one gear is adjacent to another gear, the driving mechanism including
a gear in the plurality of gears having plurality of teeth;
another gear in the plurality of gears having a plurality of teeth, said plurality of teeth of said another gear interact with said plurality of teeth of said gear;
wherein said gear and said another gear include helical gears to cause reduction of noise associated with operation of the driving mechanism.
